(19) United States
(12) Patent Application Publication
(10) Pub. No.: US 2026/0331540 A1
(43) Pub. Date: Oct. 22, 2026

(54) ON-SITE ELECTRICITY GENERATION WHICH IS NOT CONNECTED TO AN ELECTRICAL GRID FROM METHANE OR METHANOL AND HAS CARBON DIOXIDE CIRCULARITY

(71) Applicant: Technip Energies France, Nanterre Cedex (FR)

(72) Inventors: Audrey LOPEZ, NANTERRE CEDEX (FR); Estefany BARRON, NANTERRE CEDEX (FR); Fabrice MARCEL, NANTERRE CEDEX (FR); Elvire CHOLLET, NANTERRE CEDEX (FR)

(21) Appl. No.: 19/113,288

(22) PCT Filed: Sep. 27, 2023
(86) PCT No.: PCT/EP2023/076776

§ 371 Date: Mar. 19, 2025

(30) Foreign Application Priority Data
Sep. 27, 2022 (FR) FR2209787

(51) Int. Cl. H01M 8/04082; H01M 8/04089; H01M 8/0612; H01M 8/0656; H01M 8/1011

(52) U.S. Cl. CPC H01M 8/04208; H01M 8/04097; H01M 8/0618; H01M 8/0656; H01M 8/1011; H01M 2250/20

(57) ABSTRACT

An electricity generation plant, comprising two units and, a first unit and a second unit, which are located on two separate industrial sites having a first unit comprising a synthesis device which is capable of producing methane or methanol from hydrogen and carbon dioxide originating from a second unit, and the second unit comprising a fuel cell device which is capable of supplying an electric current from methane or methanol originating from the first unit and an anode gas stream comprising carbon dioxide, the fuel cell device being combined with a collecting device for collecting the carbon dioxide in the anode stream that is intended for the first unit.

The present invention relates to a plant for generating electricity in a unit, hereinafter referred to as the “second unit”, from methane or methanol produced in another unit, hereinafter referred to as the “first unit”, with carbon dioxide recovery, the two units being located at two separate industrial sites.

In particular, the invention will apply to the case of a first unit connected to the conventional power grid (continental metropolitan network), preferably a terrestrial unit, and a second unit not connected to the conventional power grid.

Examples of units not connected to the conventional power grid include those located in water (sea, lake or river), in off-grid rural areas, on islands, etc. and/or all mobile units.

For example, on a water-based unit, many auxiliary elements consume energy: Refrigerators, navigation lights, anchor lights, VHF, interior lighting, navigation aids, water pumps, etc. This energy, necessary for safety and comfort, is drawn from the on-board batteries, which therefore need to be recharged regularly.

A water-based unit, such as a boat, will need a great deal of energy in terms of motor power.

One of the solutions available for generating this energy and recharging the batteries needed for auxiliaries is the use of fuel cells.

A fuel cell generates an electric current through an electrochemical reaction between a combustible fluid and the oxygen in the air. The fuel cell typically consists of one or more Solid Oxide Fuel Cells (SOFCs).

These cells are designed mainly for stationary applications, with output power ranging from 1 KW to 2 MW. In the context of the invention, the fuel cell device will preferably provide an output power ranging from 15 MW to 100 MW.

An SOFC element is generally made up of four layers, three of which are ceramic. A single cell made up of these four superimposed layers is typically just a few millimeters thick. Dozens of these cells are then superimposed in series to form a stack.

In these cells, oxygen ions formed on the cathode side are moved through a solid oxide used as a high-temperature electrolyte to react with the fuel gas, such as hydrogen, on the anode side.

This electrochemical reaction leads to the production of electricity, as well as the formation of carbon dioxide and water, the by-product of the electrochemical reaction.

However, carbon dioxide CO2 is one of the main greenhouse gases whose release into the atmosphere contributes to global warming. To reduce the environmental impact of fuel cells, it is therefore necessary to minimize carbon dioxide emissions into the atmosphere.

This raises the problem of how to provide an electricity generation plant with reduced environmental impact.

One solution of the present invention is an electricity generation plant 1, comprising two units, a first unit A and a second unit B, located on two separate industrial sites having:
the first unit A comprising a synthesis device 8 which is capable of producing methane or methanol 15 from hydrogen 2 and carbon dioxide 4 originating from the second unit B, and
the second unit B comprising a fuel cell device 5 which is capable of supplying an electric current 1 from methane or methanol 15 originating from the first unit A and an anode gas stream 6 comprising carbon dioxide, said fuel cell device being combined with a collecting device 7 for collecting the carbon dioxide 17 in the anode stream 6 that is intended for the first unit A.

The plant according to the invention may comprise one or more of the following features, considered alone or according to any technically possible combination:
the first unit is connected to the main power grid and the second unit is not connected to the main power grid;
the first unit comprises an electrolyzer capable of producing hydrogen from water and electricity, and means for liquefying methane or methanol;
the second unit comprises, upstream of the fuel cell device, means for storing liquid methane or liquid methanol originating from the first unit and means for gasifying the liquid methane or liquid methanol;
the second unit comprises means for liquefying carbon dioxide originating from the carbon dioxide collecting device;
the second unit is chosen from a boat or a fixed or floating platform.

Another object of the invention is an electricity generation process using a plant as defined above and comprising the following steps:
a) On the first unit, a step of producing methane or methanol from hydrogen and carbon dioxide originating from the second unit,
b) On the second unit, a step of producing electricity and an anode gas stream comprising carbon dioxide by means of the fuel cell device from methane or methanol originating from the first unit, and
c) On the second unit, a step of collecting carbon dioxide to form a carbon dioxide gas stream used in step a) and a carbon dioxide-depleted anode stream.

The method according to the invention may comprise one or more of the following features, considered alone or according to any technically possible combination:
the method comprises, upstream of step a), a step of producing hydrogen by electrolysis, carried out on the first unit;
the method comprises between steps a) and b):
A step of liquefying the methanol or methane produced in step a), carried out on the first unit,
A step of storing the liquefied methanol or liquefied methane, carried out on the second unit, and
A step of gasifying the stored methanol or methane, carried out on the second unit;
the method comprises, between steps b) and c), a step of cooling and condensing the anode stream comprising carbon dioxide so as to dry said anode stream;
the method comprises, downstream of step c), a step of recycling the carbon dioxide-depleted anode stream into the fuel cell device;
downstream of step c), the method comprises a step of liquefying the carbon dioxide gas stream;
in step c) the anode stream is brought into contact with a liquid solvent capable of absorbing carbon dioxide to form the carbon dioxide-depleted anode stream and a bottom liquid stream comprising the carbon dioxide-loaded liquid solvent.

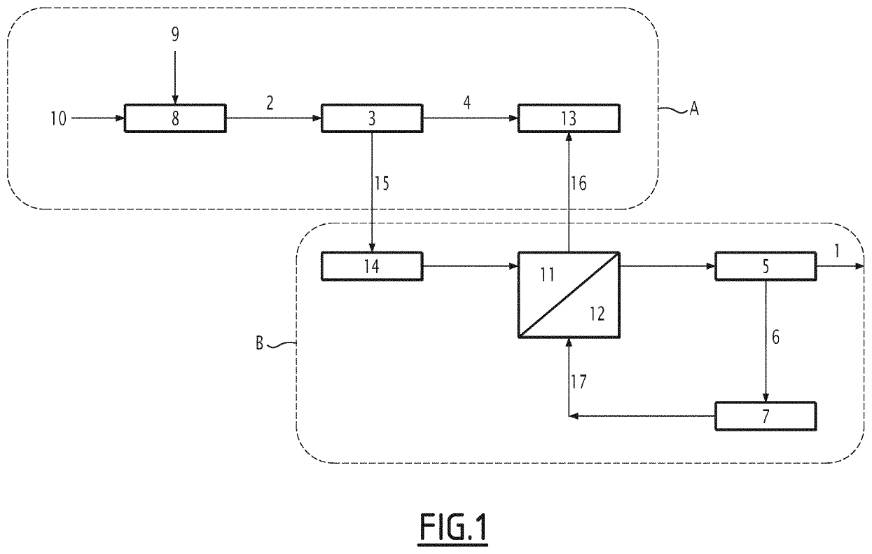

FIG. 1 shows a diagram of the plant according to the invention.

The synthesis device is preferably a “synthesis reactor”.

“Two distinct industrial sites” preferably means two sites separated from each other by a distance of more than 30 km, preferably more than 100 km, even more preferably a distance of several hundred kilometers.

Thus, the invention consists in coupling a first unit for synthesizing a fuel, in this case methane or methanol, with a second unit for generating electricity.

Synthetic methanol can be produced by various methods:
Thermocatalytic Methanation: Hydrogen and CO2 are pressurized and mixed before being sent to a series of catalytic reactors to produce synthetic methane
Methanation by Electrochemical Reduction: Water and CO2 are injected into an electrolyzer to produce synthetic methane directly. Alternatively, this method can be carried out in two stages: a co-electrolysis stage in which water and CO2 are transformed into CO and H2 in a SOEC, the CO and H2 then being thermocatalytically transformed into CH4.
Biological methanation: Synthetic methane produced biologically, i.e. using micro-organisms. The H2 can be sent directly to a methanizer to transform the CO2 in-situ. Alternatively, a dedicated biological reactor is used, which is fed with H2 and CO2 to produce methane.

The synthesis of methanol from hydrogen and carbon dioxide is represented by three macroscopic reactions:

Reaction 1: CO₂+3H₂→CH₃OH+H₂O

Reaction 2: CO2+H₂→CO+H₂O

Reaction 3: CO+2H₂→CH₃OH

CO2 reacts with hydrogen to produce methanol and water, in reaction 1. It is accompanied by reaction 2, the reverse water-gas shift (RWGS), which consumes the same reagents, but is not desirable for our purposes. The production of CO by RWGS opens up an additional pathway for methanol synthesis: Hydrogenation of CO (reaction 3).

Methanol synthesis is carried out on one or more types of solid catalysts, preferably copper-based catalysts. Among them, Cu/ZnO/Al2O3 is the conventional catalyst thanks to its excellent activity and low price compared with others. When copper is supported on alumina, methanol selectivity is highest, while the highest methanol yield was observed on zinc oxide. They showed that alumina improves methanol selectivity, as it facilitates the reduction of copper species thought to be responsible for methanol synthesis.

Methanol synthesis will preferably be carried out in a tubular fixed-bed reactor, preferably a tubular reactor similar to that described in FR3103714.

The principle behind the method for producing methanol from CO2 is quite simple. The H2/CO2 mixture, preferably preheated, is sent to the reactor, whose output stream is directed to a separation section where the methanol (accompanied by water and low percentages of higher-order alcohols) is condensed. Unreacted gases are returned to the reactor to maximize yield, as conversion is often around 20%. The methanol is finally purified by distillation.

The synthesis of methane, known as the Sabatier reaction (Sabatier 1902), is carried out using carbon dioxide and dihydrogen:

Reaction 4: CO2+4H2→CH4+2H2O

This reaction takes place in the presence of a catalyst, preferably nickel. Conversion of CO2 to CH4 is negligible at temperatures below 200 °C. Methane synthesis is best carried out at temperatures between 250 and 700 °C.

Like methanol synthesis, methane synthesis will preferably be carried out in a tubular fixed-bed reactor, preferably a tubular reactor similar to that described in FR3103714.

The principle behind the method of producing methane from CO2 is similar to that of methanol production. The H2/CO2 mixture is sent to the reactor, whose outlet flow is directed to a separation section where the methane is condensed. Unreacted gases are returned to the reactor to maximize yield. The methanol is finally purified by distillation.

The fuel cell device consists of one or more solid oxide fuel cells (SOFC). Each fuel cell is fed continuously with the methane or methanol stream injected at the anode system (partial reforming takes place before injection at the anodes) and with an oxygen-rich gas stream injected at the cathode system.

Depending on the case, the plant according to the invention may have one or more of the following features:
the first unit A is connected to the main power grid and the second unit is not connected B to the main power grid. As previously mentioned, examples of units not connected to the main power grid include those located in water (sea, lake or river), in off-grid rural areas, on islands, etc. and/or all mobile units.
the first unit A and the second unit B are two land-based units;
the first unit A is a fixed unit, preferably a land-based unit, and the second unit B is a mobile unit, preferably a water-based unit;
the second unit is chosen from a boat or a fixed or floating platform. A fixed platform is a platform anchored to the seabed. In the case of a boat, the electricity supplied by the fuel cell device can be used as motive power.
the first unit A comprises an electrolyzer 8 capable of producing hydrogen 2 from water 9 and electricity 10, and means for liquefying methane or methanol 15. Preferably, electricity will come from a renewable energy source. Preferably, the methane is liquefied through a series of cryogenic cycles. Alternatively, methane can also be liquefied in an “open cycle”, i.e. by passing the methane itself through compressors and coolers so that it undergoes expansion to generate the necessary cold.
the second unit B comprises, upstream of the fuel cell device 5, means 14 for storing liquid methane or liquid methanol originating from the first unit A and means for gasifying 11 the liquid methane or liquid methanol.
the second unit B comprises means 12 for liquefying carbon dioxide 17 originating from the carbon dioxide collecting device 7; The second unit will preferably comprise a compressor between the carbon dioxide collecting device 7 and the liquefying means 12. The liquefied carbon dioxide 16 can then be stored on the second unit B before being sent to the first unit A, then stored on this first unit A in a storage means 13 and finally used in the methane or methanol synthesis device 8.
The means 11 for gasifying liquid methane or liquid methanol and the means 12 for liquefying carbon dioxide are combined into a single heat exchange device. The carbon dioxide transfers its heat to the liquid methane or methanol and vice versa, so the carbon dioxide is liquefied while the methane or methanol is gasified.
the second unit comprises an after-treatment, cooling and condensation device upstream of the carbon dioxide collecting device 7, designed to cool and dry the anode gas stream 6 to form a dry anode stream.
the carbon dioxide collecting device 7 comprises:
an absorber designed to bring the dry anode stream in contact with a liquid solvent capable of absorbing carbon dioxide to form the carbon dioxide-depleted anode stream at the top of the absorber and a bottom liquid stream comprising the carbon dioxide-loaded liquid solvent at the bottom of the absorber,
at least one heat exchanger system for heating the bottom liquid stream to form a heated bottom stream,
a vessel connected to said at least one heat exchanger system designed to form, at the top of the vessel, the carbon dioxide stream and, at the bottom of the vessel, a partially regenerated liquid solvent stream designed to be injected into the absorber.
The carbon dioxide collecting system has no stripping column.

The electrolyzer will preferably be powered by a low-carbon energy source, preferably a renewable energy source. In the electrolyzer, at the anode, water (H2O) is dissociated by the electric current to form oxygen (O2) and H+ ions; at the cathode, H+ ions recombine to form hydrogen (H2). This hydrogen is said to be “green” when it is obtained from decarbonized energy.

The electrolyzer will preferably be a proton exchange membrane (PEM) or a high-temperature electrolyzer (like a SOEC) or an alkaline electrolyzer.

Preferably, the after-treatment, cooling and condensation device also converts at least some of the carbon monoxide present in the anode gas stream into hydrogen and carbon dioxide, typically by reaction with steam. This conversion then limits oxidation of the liquid solvent used in the carbon dioxide collecting device by oxidizing carbon monoxide molecules, and increases the carbon dioxide concentration in the anode gas stream to facilitate its collection in the collecting device.

The plant according to the invention will preferably include a means for recovering an oxygen-depleted gas stream. This oxygen-depleted gas stream will be after-treated and/or discharged into the atmosphere.

A desulfurization step will not be necessary upstream of the fuel cell, as synthesis gases (methane or methanol) do not contain any.

Another object of the present invention is a method for generating electricity offshore using a plant according to the invention and comprising the following steps:
a) On the first unit A, a step of producing methane or methanol 15 from hydrogen 2 and carbon dioxide 4 originating from the second unit B,
b) On the second unit B, a step of producing electricity 1 and an anode gas stream 6 comprising carbon dioxide by means of the fuel cell device from methane or methanol 15 originating from the first unit and
c) On the second unit B, a step 7 of collecting carbon dioxide to form a carbon dioxide gas stream 17 used in step a) and a carbon dioxide-depleted anode stream.

The method according to the invention may have one or more of the following features:
It comprises, upstream of step a), a step of producing hydrogen by electrolysis 8, carried out on the first unit A;
It comprises between steps a) and b):
A step of liquefying the methanol or methane produced in step a), carried out on the land-based unit,
A step of storing 14 the liquefied methanol or liquefied methane, carried out on the sea-based unit, and
A step of gasifying 11 the stored methanol or methane, carried out on the sea-based unit.
It comprises, between steps b) and c), a step of cooling and condensing the anode stream comprising carbon dioxide so as to dry said anode stream;
It comprises, downstream of step c), a step of recycling the carbon dioxide-depleted anode stream into the fuel cell device;
Downstream of step c), it comprises a step 12 of liquefying the carbon dioxide gas stream 17. Downstream of the collecting device 7, the carbon dioxide gas stream 17 is compressed to a pressure of between 15 and 40 bar, then cooled by heat exchange with liquid methane or liquid methanol to a temperature of between −15 and −50 °C before being expanded to reach liquid CO2 storage conditions (for example: 17 bar and −28 °C). Liquid CO2 16 can initially be stored on the second unit and then on the first unit in storage means 13 before being used in the methane or methanol synthesis device 8;
The step 11 of gasifying methane or methanol is carried out by heat exchange with the thermal gas stream;
the carbon dioxide-depleted anode stream has a carbon dioxide content of 0% to 80% by volume, preferably 20% to 70% by volume, even more preferably 40% to 60% by volume;
in step c) the anode stream is brought into contact with a liquid solvent capable of absorbing carbon dioxide to form the carbon dioxide-depleted anode stream and a bottom liquid stream comprising the carbon dioxide-loaded liquid solvent;
carbon dioxide absorbed in the bottom liquid stream is released by heating the bottom liquid stream to form part of the carbon dioxide stream used in step a) and a partially regenerated liquid solvent stream;
before being injected into the fuel cell device 5, the methane or methanol is heated by heat exchange with the carbon dioxide gas stream;
at least 80% of the carbon dioxide produced during the operation of the method is recovered in the carbon dioxide gas stream, preferably at least 90 mol %, more preferably from 90% to 99% mol %.

Preferably, the plant according to the invention will further comprise a closed heat exchange circuit comprising a heat transfer fluid intended to be placed in heat exchange on the one hand with the bottom liquid stream comprising the carbon dioxide-loaded liquid solvent, at said at least one heat exchanger system, and on the other hand with the anode gas stream and/or the cathode gas stream.

Advantageously, the methane or methanol introduced into the fuel cell device will be at a pressure of between atmospheric pressure and 2 bar, preferably at a temperature of between 300 °C and 500 °C.

The dioxygen-rich gas stream typically has a dioxygen content of at least 10% by volume, preferably 15% to 25% by volume of dioxygen. Advantageously, the dioxygen-rich gas stream is an air stream.

Preferably, the dioxygen-rich gas stream is at a pressure of between atmospheric pressure and 2 bar.

Preferably, the liquid solvent used in the collecting device is chosen from aqueous amine solutions capable of adsorbing carbon dioxide.

Chemical absorption methods using aqueous amine solutions capable of absorbing carbon dioxide are well known to those skilled in the art and will not be described further here. Examples of aqueous amine solutions suitable for the invention include solutions composed of one or more of the following compounds: monoethanolamine, diethanolamine, N-methyldiethanolamine, piperazine, 2-Amino-2-methylpropan-1-ol, Bis(2-hydroxypropyl)amine, 1-methylpiperazine, dimethylaminoethanol.

A number of equipment and utility systems required for the proper operation of the absorption units are known to those skilled in the art and will therefore not be described further in the remainder of the description.

On contact with the dry anode stream, the liquid solvent absorbs the carbon dioxide present in the dry anode stream to form a bottom liquid stream made up of the carbon dioxide-laden liquid solvent and, at the top of the absorber 200, the carbon dioxide-depleted anode stream.

The carbon dioxide-depleted anode stream typically comprises from 10% to 60% by volume of carbon dioxide CO2, preferably from 30% to 50% by volume.

The bottom liquid stream consisting of the carbon dioxide-loaded liquid solvent is then heated to form a preheated bottom stream. The preheated bottom stream 206 is then heated again so that the carbon dioxide absorbed in the liquid solvent is partially released in gaseous form. Typically, the preheated bottom stream is heated, preferably by heat exchange, to a temperature above 70 °C, preferably above 80 °C, particularly close to 90 °C. The heated bottom stream is introduced into the vessel to form the carbon dioxide stream at the top of the vessel, and a carbon dioxide-depleted liquid solvent stream at the bottom of the vessel.

The carbon dioxide-depleted liquid solvent stream is then re-injected into the absorber for recycling.

The carbon dioxide-depleted liquid solvent stream taken from the bottom of the vessel then comprises the starting liquid solvent and some of the absorbed carbon dioxide not released by the expansion in the vessel 216.

Typically, at least 15% of the liquid solvent is still loaded with carbon dioxide, preferably at least 35%, more preferably 55 to 65%.

In other words, the carbon dioxide-depleted liquid solvent stream has a carbon dioxide content greater than or equal to 0.1 moles of CO2 per mole of amino solvent on a dry basis, preferably from 0.15 to 0.4 moles of CO2 per mole of amino solvent on a dry basis, even more preferably from 0.30 to 0.35 moles of CO2 per mole of amino solvent on a dry basis.

Preferably, before being re-injected into the absorber, the carbon dioxide-depleted liquid solvent stream is cooled, preferably by heat exchange with the bottom liquid stream coming from the absorber.

The carbon dioxide-depleted liquid solvent stream can also undergo an additional cooling step before being injected into the absorber, for example by passing through a heat exchanger using an external refrigerant source.

In the solution proposed by the present invention, carbon circularity is implemented: the carbon dioxide produced by the fuel cell is reused in the methane or methanol synthesis device.

From there, the main benefits are the reduction of the carbon footprint linked to the recycling of carbon dioxide. As the quantities of CO2 required for methanation are significant, and resources of CO2 of biogenic origin are limited, CO2 circularity helps to reduce emissions in the chain.

1. An electricity generation plant, comprising two units, a first unit and a second unit, which are located on two separate industrial sites having:
the first unit comprising a synthesis device which is capable of producing methane or methanol from hydrogen and carbon dioxide originating from the second unit; and,
the second unit comprising a fuel cell device which is capable of supplying an electric current from methane or methanol originating from the first unit and an anode gas stream comprising carbon dioxide, said fuel cell device being combined with a collecting device for collecting the carbon dioxide in the anode stream that is intended for the first unit

2. The electricity generation plant according to claim 1, wherein the first unit is connected to the main power grid and the second unit is not connected to the main power grid.

3. The electricity generation plant according to claim 1, wherein the first unit comprises an electrolyzer capable of producing hydrogen from water and electricity, and a methane or methanol liquefying means.

4. The electricity generation plant according to claim 1, wherein the second unit comprises, upstream of the fuel cell device, a means for storing liquid methane or liquid methanol from the first unit and a means for gasifying the liquid methane or liquid methanol.

5. The electricity generation plant according to claim 1, wherein the second unit comprises means for liquefying carbon dioxide originating from the carbon dioxide collecting device.

6. The offshore electricity generation plant according to claim 1, wherein the second unit is selected from a boat or a fixed or floating platform.

7. A method for generating electricity using the plant as defined in claim 1 and comprising the following steps:
a) the first unit, a step of producing methane or methanol from hydrogen and carbon dioxide originating from the second unit;
b) on the second unit, a step of producing electricity and an anode gas stream comprising carbon dioxide by means of the fuel cell device from methane or methanol originating from the first unit; and,
c) on the second unit, a step of collecting carbon dioxide to form a carbon dioxide gas stream used in step a) and a carbon dioxide-depleted anode stream.

8. The method for generating electricity according to claim 7, wherein it comprises, upstream of step a), a hydrogen production step by electrolysis, carried out on the first unit.

9. The method for generating electricity according to claim 7, it comprises between steps a) and b):
a step of liquefying the methanol or methane produced in step a), carried out on the first unit;
a step of storing the liquefied methanol or liquefied methane, carried out on the second unit; and,
a step of gasifying the stored methanol or methane, carried out on the second unit.

10. The method for generating electricity according to claim 7, wherein it comprises between steps b) and c) a step of cooling and condensing the anode stream comprising carbon dioxide so as to dry said anode stream.

11. The method for generating electricity according to claim 7, wherein it comprises, downstream of step c), a step of recycling the carbon dioxide-depleted anode stream into the fuel cell device.

12. The method for generating electricity according to claim 7, wherein it comprises downstream of step c) a step of liquefying the carbon dioxide gas stream.

13. The method for generating electricity according to claim 7, wherein the carbon dioxide-depleted anode stream has a carbon dioxide content of 0% to 80% by volume, preferably 20% to 70% by volume, even more preferably 40% to 60% by volume.

14. The method for generating electricity according to claim 7, wherein in step c) the anode stream is contacted with a liquid solvent capable of absorbing carbon dioxide to form the carbon dioxide-depleted anode stream and a bottom liquid stream comprising the carbon dioxide-loaded liquid solvent.

15. The method for generating electricity according to claim 7, wherein at least 80% of the carbon dioxide produced during the operation of the method is recovered in the carbon dioxide gas stream, preferably at least 90 mol %, more preferably from 90% to 99% mol %.
